SEQUENCE LISTING (20 sequences)

SEQ ID NO: 1 - RNA, 7 nt, Artificial Sequence
  unsure 6: pox virus; n is g,a,u or c
uuuuunu

SEQ ID NO: 2 - DNA, 7 bp, artificial sequence
  nascent pox virus termination signal
ttttttt

SEQ ID NO: 3 - RNA, 22 nt, artificial sequence
  unsure 14: U5NU-22-mer oligo; n is g,a,u or c
gggccggcuuuuunuuugcguu

SEQ ID NO: 4 - RNA, 22 nt, artificial sequence
  mutant- 22-mer oligo
gggccggcaauuauuaugcguu

SEQ ID NO: 5 - RNA, 36 nt, artificial sequence
  unsure 23: pGEM-U5NU-36-mer oligo n is g,a,u or c
gggcgaauugggccggcuuuuunuuugcguugaauu

SEQ ID NO: 6 - RNA, 36 nt, artificial sequence
  pGEM-Br-U5NU-36-mer; uracils at 8,9,18-26, 30, 31, 35 and 36 are brominated
gggcgaauugggccggcuuuuuuuuugcguugaauu

SEQ ID NO: 7 - DNA, 27 bp, artificial sequence
  transcription template plasmid oilgo
aattgggccggctttttttttgcgttg

SEQ ID NO: 8 - DNA, 22 bp, artificial sequence
  dU5NdU oligo; 9-17 is deoxyuracil
gggccggcuuuuuuuuugcguu

SEQ ID NO: 9 - RNA, 22 nt, artificial sequence
  BrdU5NdU oligo; 9-17 is brominated deoxyuracil
gggccggcuuuuuuuuugcguu

SEQ ID NO: 10 - DNA, 22 bp, artificial sequence
  T5NT
gggccggctttttttttgcguu

SEQ ID NO: 11 - DNA, 22 bp, artificial sequence
  mutant T5NT
gggccggcaattattatgcgtt

SEQ ID NO: 12 - RNA, 17 nt, artificial sequence
  unsure 10: U5NU 17-mer oligo; n is a,g,u or c
cggcuuuuunuuugcgu

SEQ ID NO: 13 - RNA, 13 nt, artificial sequence
  unsure 8: U5NU-13-mer oligo; n is a,g, u or c
gcuuuuunuuugc

SEQ ID NO: 14 - RNA, 9 nt, artificial sequence
  unsure 6: U5NU-9-mer oligo; n is a,g,u or c
uuuuunuuu

SEQ ID NO: 15 - RNA, 7 nt, artificial sequence
  unsure 6: U5NU-7-mer oligo; n is a,g,u or c
uuuuunu

SEQ ID NO: 16 - RNA, 7 nt, artificial sequence
  mutant 7-mer
uuuuuau

SEQ ID NO: 17 - DNA, 22 bp, artificial sequence
  unsure 14: DNA-U9-DNA oligo; n is g,a,u or c; 1-8 and 18-22 are DNA
gggccggcuuuuunuuugcguu

SEQ ID NO: 18 - RNA, 22 nt, artificial sequence
  unsure 14: 2-prime-O-Me-RNA-PT-U9-2-prime-O-Me-PT-RNA oligo; n is g,a,u or c; 1-8 and 18-22 are 2-prime-O methylated; 1-8 and 17-22 have phoshporthiol bonds
gggccggcuuuuunuuugcguu

SEQ ID NO: 19 - DNA, 22 bp, artificial sequence
  unsure 14: PT-DNA-U9-PT-DNA oligo; n is g,a,u or c; 1-8 and 18-22 are phosphorthiols; 9-17 are RNA
gggccggcuuuuunuuugcguu

SEQ ID NO: 20 - RNA, 7 nt, artificial sequence
  U5AU oligo
uuuuauu

(19) United States
(12) Patent Application Publication
(10) Pub. No.: US 2005/0085435 A1
(43) Pub. Date: Apr. 21, 2005

(54) Novel inhibitors of poxvirus replication

(76) Inventors: Edward G. Niles, Amherst, NY (US); Mohamed R. Mohamed, Amherst, NY (US)

Correspondence Address: HODGSON RUSS LLP, ONE M & T PLAZA, SUITE 2000, BUFFALO, NY, 14203-2391, US

(21) Appl. No.: 10/750,939

(22) Filed: Jan. 2, 2004

Related U.S. Application Data
(60) Provisional application No. 60/437,945, filed on Jan. 2, 2003.

(51) Int. Cl. A61K 48/00; C07H 21/02; C07H 21/04

(52) U.S. Cl. 514/44; 536/23.1

(57) ABSTRACT

The present invention provides compositions and methods for generation of premature transcription termination products. Premature termination of transcription interferes with the normal transcription and termination of early phase genes of the poxviruses and results in inhibition of replication of the poxviruses. The compositions of the present invention are oligonucleotides comprising a U5NU sequence flanked by ribonucleotides, deoxyribonucleotides or modifications thereof. This invention also provides methods for inhibition of replication of poxvirus including the small poxvirus, Monkeypox virus and vaccinia virus.

This application claims priority to U.S. Provisional Application Ser. No. 60/437,945, filed Jan. 2, 2003, the disclosure of which is incorporated herein by reference.

This work was supported by grant no. RO1-AI 43933 from the National Institute of Allergy and Infectious Diseases. The government has certain rights in the invention.

### FIELD OF THE INVENTION

This invention relates generally to the area of poxviruses and more particularly to compositions and methods for inhibition of poxvirus replication.

### BACKGROUND OF THE INVENTION

Variola, a form of poxvirus, is the causative agent for smallpox and is considered to be among the most virulent human pathogens of all time. Due to the existence of an efficacious live vaccine in the form of another poxvirus, vaccinia, and the perseverance of a cadre of dedicated health professionals, smallpox was declared eradicated from the natural population in 1980. However, laboratory stocks of smallpox were collected and stored in some laboratories, stimulating a long debate on the value of maintaining stocks of this virulent pathogen. Now, faced with an ever increasing naïve population and declining immunity in the older generation who were vaccinated as children, combined with the threat of poxviruses re-emerging through bio-terrorism or by a natural occurrence, the possibility of large scale vaccination is being actively debated.

Poxviruses are double stranded DNA viruses that replicate in the cytoplasm of infected cells. In order to conduct this unusual life cycle, poxviruses encode the enzymes employed in viral gene transcription, mRNA processing, genome replication and recombination (1). Poxvirus gene expression is divided into three temporal classes that differ in their promoter sequences and the protein factors employed in transcription initiation (2). Early genes are transcribed in the virion core by a virus encoded multi-subunit RNA polymerase (3) that contains the Rap 94 subunit, the product of the H4L gene (4,5). Early messages are capped (6) and polyadenylylated (7) by virus encoded enzymes. Shut off of early gene transcription accompanies the onset of DNA replication. Among the early gene products are proteins that direct intermediate gene transcription (8,9). Late gene expression follows the accumulation of intermediate gene products, including late gene transcription factors (10). Both intermediate and late gene transcription require a replicating template and also employ one or more host encoded transcription factors (11-13).

Unique among viral gene classes, early gene transcription termination is subject to signals and factor dependent transcription termination (14). Termination requires the virion form of RNA polymerase, containing the Rap 94 subunit (15), and VTF (16), the vaccinia termination factor composed of the 97 kDa D1R subunit (17) and the 33 kDa D12L subunit (18). VTF is also the virion mRNA capping enzyme employed in catalyzing the first three steps in cap formation (6,19). In addition, ATPase activity catalyzed by nucleoside triphosphate phosphohydrolase I (NPH I), the product of gene D11L, is essential for transcription termination and transcript release (20,21). An interaction between the C-terminal end of NPH I and the N-terminal end of Rap 94 is required for termination (15,22-24). Finally, termination also utilizes a sequence present in the gene about 30 to 50 base pairs upstream from the map position of the early mRNA poly A addition site which is recognized in the nascent mRNA (25). The sequences of Rap 94, VTF and NRH I have greater than 90% homology across the orthopox genus, which includes small pox, cow pox, mouse pox, rabbit pox, monkey pox and other virulent poxvirus species.

Although vaccinia virus is an efficacious live virus vaccine, which provides protective immunity in all who exhibit a valid “take”, vaccinia virus also elicits significant complications in a portion of the vaccinees. Even in healthy individuals, the size and severity of the lesion produced at the site of inoculation is notable. Furthermore, infectious virus can be spread from the site of vaccination until the wound scabs over, raising the specter of unintended virus spread. With the expectation of 1/1,000,000 deaths among healthy vaccinees and a far greater proportion among the immune compromised individuals, the need for efficacious anti-poxvirus therapeutic agents is apparent.

### SUMMARY OF THE INVENTION

The present invention provides methods and compositions useful for inhibiting replication of poxviruses. The compositions of the present invention comprise oligonucleotides of between 8 to about 40 nucleotides comprising an oligoribonucleotide portion which has the sequence UUUUUNU (SEQ ID NO:1), where N is any ribonucleotide. This sequence is also referred to as “U5NU” hereinafter. In one embodiment, one or more nucleotides flank one or both ends of the U5NU sequence (referred to herein as the “flanking regions” or “flanking portions”). The flanking regions may comprise nucleotides other than ribonucleotides, such as deoxyribonucleotides, modified deoxyribonucleotides and modified ribonucleotides. The modifications in the ribonucleotide is generally at the 2′ position of the ribose and includes 2′O-methyl, 2′-O-(2-Methoxyethyl), and 2′O-(2-Aminopropyl), and combinations thereof. The flanking portions of the oligonucleotides of the present invention may comprise phosphodiester linkages, or synthetic inter-nucleoside linkages, such as phosphorothiols, methylphosphonates, phosphoramidites, or morpholinos.

The present invention also provides a method for inhibition of poxvirus replication in a cell. The method comprises the step of providing to the cell oligonucleotides of between 8 and about 40 nucleotides comprising the U5NU sequence.

The present invention also provides a method for inhibition of poxvirus replication in an individual. The method comprises the step of administering to the individual a composition comprising one or more oligonucleotides of the present invention by any suitable route of administration. For example, the method of the present invention is useful for administrating the compositions of the present invention to individuals exposed to a poxvirus including, but not limited to, the smallpox virus, and to individuals who are at risk of contact with the poxvirus. The present compositions may also be administered to individuals who have an adverse reaction to vaccination to smallpox.

The compositions and methods of the present invention are not limited to the small poxvirus and can also be used in animals that have been infected with a poxvirus or at risk of coming in contact with a poxvirus.

### BRIEF DESCRIPTION OF THE DRAWINGS

FIG. 1 is a map of the bead bound G21(TER29)A78 DNA template. Arrows represent the RNA products synthesized under various experimental reaction conditions. The lengths of the RNA products are noted on the right. RT indicates the full length read through transcript of 177 nucleotides; Term indicates termination product; Step 1 indicates RNA made in the presence of ATP, CTP and UTP and 3′O-methyl-GTP; Step 2 indicates RNA made by elongating the Step 1 (G21RNA) in the presence of UTP, CTP, GTP and cordycepin triphosphate. The Ter 29 template is 324 bp long, has a promoter (Pr), a G-less cassette, an A-less cassette and biotin (B). A 20-nucleotide G-less cassette (G-less) is followed by three G residues (G3) at positions +21 to +23. A termination signal, TTTTTTTTT, (SEQ ID NO:2), lies within the A-less cassette, spanning position +29 to +37. Four adenine residues (A4) downstream of the G-less cassette are at positions +78 to +81.

FIG. 2 is a representation of an autoradiograph showing transcripts produced off the Ter 29 template in the absence or presence of various concentrations of an oligonucleotide comprising the U5NU sequence (SEQ ID NO:3; U5NU-22-mer) wherein N was U. VTF was included in the transcription reaction (lanes 3-9). RT indicates read through products, Term indicates termination products and PT indicates premature termination products. The % PT, indicated for lanes 2-9, was quantified by scanning the autoradiograph with a densitometer; P and U9 P indicate pause products. G 21 was run in lanes 1 and 10 as a size marker. The data shows that the U5NU-22-mer oligonucleotide mediates the premature termination of transcription.

FIG. 3 is a representation of an autoradiograph showing transcripts produced off the Ter 29 template in the absence or presence of increasing concentrations of an oligonucleotide which does not comprise the U5NU sequence (SEQ ID NO:4; mutant-22-mer). Abbreviations are the same as indicated in FIG. 2 except that % Termination indicates the percent of premature termination products. The data shows that the mutant-22-mer oligonucleotide does not mediate premature termination of early gene transcription.

FIGS. 4A and 4B are representations of autoradiographs showing transcripts produced off the Ter 29 template and the Ter 59 template, respectively, in the absence (lanes 2, 3) or presence of U5NU-22-mer oligonucleotide—indicated as “U”, wherein N was U (lane 4); or the mutant-22-mer oligonucleotide—indicated as “M” (lane 5). VTF was included in the transcription reaction (lanes 3-5). The abbreviations are the same as in FIG. 2. The data demonstrate that premature termination is observed for both the Ter 29 (FIG. 4A) and the Ter 59 (FIG. 4B) templates and is therefore independent of the template sequence.

FIG. 5A is a representation of an autoradiograph showing the release of premature termination transcripts (G21) in the absence (lanes 9, 10) or presence of the U5NU-22-mer—indicated as “U” (lanes 1, 2, 5, 6, 11, 12, 15 and 16) wherein N was U; or the mutant-22-mer—indicated as “M” (lanes 3, 4, 7, 8, 13, 14, 17 and 18). VTF, NPH I and ATP were included in the transcription reaction where indicated. B indicates bound and F indicates free or released transcripts. % Release indicates the percentage of the premature termination product released from the bead bound ternary complex. The data demonstrate that an oligonucleotide comprising the U5NU oligonucleotide stimulates the release of the G21 transcript from a bead bound ternary complex.

FIG. 5B is a representation of an autoradiograph showing the release of the premature termination product as a function of incubation time from the bead bound ternary complex in the absence or presence of the U5NU-22-mer—indicated as “U” (lanes 3-14). The abbreviations are the same as in FIG. 5A. These data demonstrate that release is a rapid process, in vitro.

FIGS. 6A, 6B and 6C are representations of autoradiographs showing transcripts produced off the Ter 29 template in the absence or presence of increasing concentrations of oligonucleotides which do not have the U5NU sequence. In FIG. 6A, T5NT (SEQ ID NO:10) was used, in FIG. 6B, dU5NdU (SEQ ID NO:8) and in FIG. 6C, BrdU5NdU (SEQ ID NO: 9) was used. Abbreviations are the same as indicated in FIGS. 2 and 3. No premature transcription termination products are observed for any of these oligonucleotides. These data further demonstrate a requirement for the U5NU sequence for premature termination.

FIGS. 7A and 7B are representations of autoradiographs showing premature transcripts produced off the Ter 29 template in the absence or presence of increasing concentrations of a 36 mer oligonucleotide, pGEM-U5NU-36-mer (SEQ ID NO:5) comprising the U5NU sequence wherein N was U, and a variant, pGEM-Br-U5NU-36-mer, in which all the uracils in the U5NU sequence were brominated (SEQ ID NO:6). Abbreviations are the same as indicated in FIGS. 2 and 3. The data demonstrate that while the 36-mer comprising the U5NU sequence generated premature transcription termination, the effect was not seen if all the uracils in the U5NU sequence were brominated.

FIG. 8 is a graphical representation of inhibition of U5NU stimulated premature termination by various mutant oligonucleotides. ●, BrdU5NdU; ▴, mutant-22mer; O, T5NT; +, dU5NdU.

FIGS. 9A, 9B and 9C are representations of autoradiographs showing the generation of premature transcription products by oligonucleotides of different lengths comprising the U5NU sequence. In FIG. 9A, a 17-mer, U5NU-17-mer (SEQ ID NO:12) was used; in FIG. 9B, a 13-mer, U5NU-13-mer (SEQ ID NO:13) was used; in FIG. 9C, a 9-mer, U5NU-9-mer (SEQ ID NO: 14) was used. The abbreviations are similar to FIG. 2.

FIG. 9D is a graphical representation of the percent of premature termination products for the oligonucleotides used in FIGS. 9A-9C and FIG. 2. Data is shown for U5NU-22-mer (●); U5NU-17-mer (▴); U5NU-13-mer (O); U5NU-9-mer (+); and U5NU-7-mer (□).

FIGS. 10A and 10B are representations of an autoradiograph showing the generation of premature transcription termination products by an oligonucleotide comprising the U5NU sequence flanked by DNA sequences. Data is shown for a chimeric 22mer comprising U5NU flanked by oligodeoxyribonucleotides with phosphodiester linkages (DNA-U9-DNA—SEQ ID NO:17) in (FIG. 10A) and an oligonucleotide comprising the U5NU sequence flanked by oligodeoxyribonucleotides with phosphorothioldiester linkages (SEQ ID NO: 19- PT-DNA-U9-PT-DNA) (FIG. 10B). The abbreviations are the same as in FIG. 2. These data demonstrate that inter-nucleoside linkages in the flanking region are not limited to phosphodiester linkages.

FIG. 11A is a representation of an autoradiograph showing a time dependent accumulation of transcription products synthesized in virus cores. Data is shown for core bound (C) and free (F) RNA separated and analyzed by gel electrophoresis followed by fluorography.

FIG. 11B is a graphical representation of results derived from the average of multiple determinations of the data presented in FIG. 11A.

FIG. 12A is a representation of an autoradiograph showing transcription products in virus cores in the absence or presence of the indicated amount of the oligonucleotide U5NU-22-mer (“U”), the mutant-22-mer (“M”), or the BrdU5NdU oligonucleotide (“B-dU). The abbreviations are the same as in FIG. 11.

FIG. 12B is a graphical representation of results derived from FIG. 12A for core bound (filled bars) and free (empty bars).

FIGS. 13A-13D are representations of autoradiographs showing transcription products produced by virus cores in the presence of increasing concentrations of oligonucleotides of various lengths comprising the U5NU sequence. C indicates core bound and F indicates Free products. Data is shown for U5NU-22-mer (FIG. 14A); U5NU-17-mer (FIG. 14B); U5NU-13-mer (FIG. 14C) and U5NU-9-mer (FIG. 14D).

FIG. 14A is a representation of an autoradiograph showing transcription products in virus core in the absence or presence of increasing amounts of DNA-PT-U9-DNA-PT demonstrating that chimeric 22mers comprising the U5NU sequences flanked with normal DNA can stimulate premature transcription termination in viral cores.

FIG. 14B is a representation of an autoradiograph showing transcription products in virus cores in the absence or presence of increasing amounts of another chimeric oligonucleotide (2′OMe-RNA-PT-U9-2′O-Me-PT-RNA; SEQ ID NO: 18) in which nucleotides in the flanking RNA sequences are modified in their sugar residues and the inter-nucleoside linkages are phosphorothiol linkages. These data further demonstrate oligonucleotides in which the flanking regions are lacking the phosphodiester linkages or in which the sugar residue is modified can generate premature transcription termination products. C, core bound RNA; F, free RNA.

### DETAILED DESCRIPTION OF THE INVENTION

The present invention provides compositions and methods useful for inhibiting replication of poxvirus. The compositions of the present invention comprise oligonucleotides comprising the sequence UUUUUNU (SEQ ID NO: 1), also referred to as “U5NU” hereinafter, where N is any of guanine, cytosine, adenine or uracil. When N is U, this sequence as referred to as “U7”. A string of nine contiguous U's are referred to herein as “U9”. The oligonucleotides of the present invention stimulate premature transcription termination of pox genes.

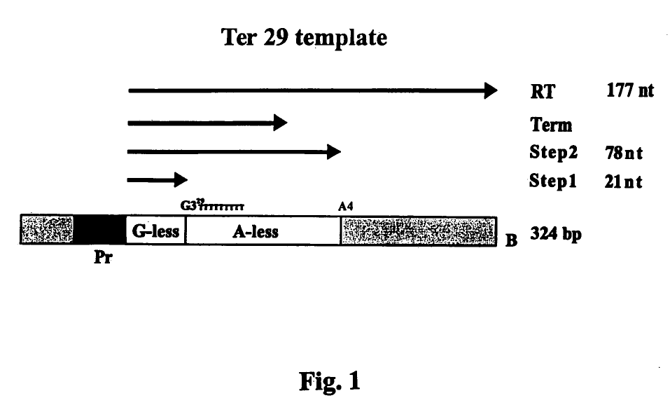

“Premature termination of transcription” or “Premature transcription termination” is used herein to indicate termination of transcription that results in RNA products which are significantly shorter than the normal transcription termination products indicative of termination of transcription of early genes in poxviruses. The normal transcription products are indicated as being about 70 bases in length in FIG. 1. The Premature transcription products in the present invention are about 21 to about 30 bases in length.

Premature transcription termination in vivo or in transcription or in transcription measures in viral cores in vitro refers to initiating the termination processes at a point prior to the usual termination point. This results in the synthesis of truncated and inactive mRNA via oligonucleotide dependent premature termination.

This oligonucleotide dependent premature termination is independent of the sequence of the transcription template and does not need the nascent viral TTTTTTTTT (SEQ ID NO:2; “T9” hereinafter) cis-acting signal which is required in normal termination of transcription in the poxvirus. Premature transcription termination will disrupt the viral life cycle and prevent viral replication. Accordingly, the oligonucleotides and methods of the present invention are useful as novel anti-poxvirus agents and methods for their administration.

The oligonucleotides of the present invention are between 8 and about 40 nucleotides in length. Preferably, they are between 8 and 36 nucleotides long, more preferably between 9 and 22 nucleotides long, and still more preferably between 9 to 13 nucleotides long. The oligonucleotides of the present invention can be synthesized by methods well known in the art. For example, run-off in vitro transcription reactions can be utilized as described in Myette, J. R., and Niles, E. G. (1996) J. Biol. Chem., 271, 11936-11944. Additionally, RNA oligonucleotides can be chemically synthesized according to methods well known to those skilled in the art. An example of a method for synthesizing RNA oligonucleotides is by using ribosephosphoramidites. In this method, the RNA amidite is reacted with tetrazole, NH₄OH is used to remove exocyclic amino protecting groups, a compound such as tetra-n-butylammonium fluoride (TBAF) is used to remove the 2′-OH alkylsilyl protecting groups, and the deprotected oligoribonucleotide is analyzed by any of a variety of well known methods, such as gel electrophoresis. Discussion of the chemical synthesis, deprotection, purification and analysis of oligoribonucleotides are provided in U.S. Pat. No. 5,831,071 and U.S. Pat. No.5,977,343. Additional examples of suitable methods for oligonucleotide purification and analysis include reverse phase or ion exchange high pressure liquid chromatography (HPLC), or hybridization affinity chromatographic methods, which are well known in the art. For examples of additional well known techniques, see Metelev and Agrawal (1992) Anal. Biochem. 200:342-346.

The oligonucleotides of the present invention comprise the U5NU sequence. In addition, flanking regions are present on the 5′ end, the 3′ end, or both ends of the U5NU sequence. The length of the flanking region is at least one nucleotide. The flanking portions of the oligonucleotides of the present invention may comprise ribonucleotides as well as other nucleotides. For example, the flanking portions may comprise deoxyribonucleotides, modified ribonucleotides and modified deoxyribonucleotides. The term “modified ribonucleotide” includes ribonucleotides having at least one nucleotide with a modified sugar, such as a 2′-O-substituted ribonucleotide. For purposes of the invention, the term “2′-O-substituted” means substitution of the 2′ position of the ribose moiety with an —O— lower alkyl group containing 1-6 saturated or unsaturated carbon atoms, or with an —O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group.

The nucleotides in the flanking regions may be linked by phosphodiester linkages or by a synthetic linkage, i.e., a linkage other than a phosphodiester linkage. Thus, the inter-nucleoside linkage in the flanking region may be phosphodiester, alkylphosphonate, phosphorothioate, phosphorodithioate, phosphate ester, alkylphosphonothioate, phosphoramidate, carbamate, carbonate, morpholino, phosphate trister, acetamidate, and/or carboxymethyl ester or combinations thereof. As for combinations of inter-nucleoside linkages, such chimeric oligonucleotides are well known in the art. For example, U.S. Pat. No. 5,149,797 discloses chimeric oligonucleotides having an oligonucleotide phosphodiester or oligonucleotide phosphorothioate core sequence flanked by oligonucleotide, methylphosphonates or phosphoramidates.

As an example of a suitable method for synthesizing chimeric oligonucleotides, U.S. Pat. No. 5,652,355 describes a method of synthesis of hybrid oligonucleotides containing segments of deoxy- and ribo nucleotides that resist nucleolytic degradation. Such oligonucleotides may have phosphorothioate and/or phosphoroithioate inter-nucleoside linkages and segments of oligodeoxyribonucleotides as well as segments of either oligoribonucleotides or 2′-substituted-oligoribonucleotides. The synthesis of hybrid oligonucleotides can be carried out by various well known procedures, including solid phase methods using phosphoramidite or H-phosphonate chemistry (see. e.g., Agrawal (1991) TIBTECH 10:152-158), and can be purified by standard techniques such as by reverse phase HPLC, ion exchange HPLC or hybridization affinity based chromatographic methods. (See, e.g., Metelev and Agrawal (1992) Anal. Biochem. 200:342-346).

Further, the oligonucleotides of the present invention may be modified at their 5′ or 3′ ends to enhance cellular uptake and prevent exonuclease digestion. One example of such a modification is the addition of cholesterol at the 5′ or 3′ ends of the oligonucleotides of the present invention.

In one embodiment, the present invention provides a method for inhibiting the replication of poxvirus. The method comprises the steps of providing to a cell in which poxvirus replication is desired, an oligonucleotide comprising a U5NU sequence. It is well known in the art that oligonucleotides can enter the cell easily. For example, see Song et al., Nature Medicine (2003) 9:347-351; McCaffrey et al., Nature (2002) 418:38-39. Thus, exposing the cells to the oligonucleotides is sufficient to effect entry into the cells.

The present invention also provides methods for inhibition of replication of poxviruses in an individual. The term “individual” as used herein includes humans as well as other mammals in which poxvirus infection is known to occur, such as cows, mice, monkeys, rabbits and the like. Accordingly, the present invention is useful for administration to individuals exposed to a poxvirus including, but not limited to, the smallpox virus or individuals who have an adverse reaction to vaccination.

To inhibit the replication of poxvirus in an individual, the oligonucleotides of the present invention may be administered by any conventional route. For example, the oligonucleotides may be administered by intravenous, intraperitoneal, intranasal, oral, transdermal, or subcutaneous routes. Such methods are well known in the art. For example, see Lewis et al., (2002) Nature Genetics, 32:107-108. The oligonucleotides of the present invention may be administered in combination with any standard pharmaceutically acceptable carriers. Suitable carriers include solvents, dispersion media, coatings, isotonic and absorption delaying agents and the like.

Effective dosages of the oligonucleotides of the present invention and modes of their administration in the treatment of poxvirus infections are dependent upon factors such as severity of infection, age and weight of the individual and are well within the purview of those skilled in the art.

The method of the present invention can also be used in animals that have been infected with a poxvirus, such as small pox, cow pox, mouse pox, rabbit pox, monkey pox and other virulent poxvirus species. For administration to animals, in addition to the routes mentioned above, the oligonucleotides may also be administered via the tail vein. For example, see Song et al., Nature Medicine (2003) 9:347-351.

The present invention also provides kits comprising compositions useful for inhibition of poxvirus replication. The kits of the present invention may comprise for example, one or more oligonucleotides which can inhibit poxvirus replication. The oligonucleotides may be present in pharmaceutically acceptable carriers. The oligonucleotides may be provided with instructions on their dosage and use.

The present invention is illustrated by the examples given below which are not meant to be illustrative and not limiting in any way.

### EXAMPLE 1

This Example demonstrates that oligonucleotides comprising a U5NU sequence stimulate premature transcription termination in poxvirus early genes.

Wild type (WT) vaccinia virus strain was propagated in BSC40 African green monkey cells at 37° C. Virus titer was determined by plaque assays on BSC40 cells at at 37° C.

Cell infection was carried out as follows. A549 cells were infected with WT virus at a multiplicity of infection (m.o.i.) of 15, at 37° C. After 24 hours, the medium was removed and replaced with 40° C. medium containing 100 μg/ml of cycloheximide. After a further 24 hours at 40° C., cells were washed and treated with 250 μg/ml lysolecithin and extracts prepared by standard methods.

The templates used to produce the RNA transcripts were constructed as described previously (26). A map of one of the templates, G21 (TER29)A78, is shown in FIG. 1. Ter 29 locates the T9 signal starting at position 29. The position of the termination signal determines the length of the termination product. The G21 (TER29)A78 transcription unit contains a synthetic early promoter fused to a 20-nucleotide G-less cassette, which is followed by three G residues at positions +21 to +23. RNA synthesis in the absence of GTP and the presence of 3′ O methyl GTP yields a 21 base product that can be identified by gel electrophoresis, Step 1, FIG. 1. A 57-nucleotide A-less cassette lies downstream of the G-less cassette and flanked at its 3′ end by four A residues at positions +78 to +81. Elongation of the Step 1 RNA in the presence of UTP, CTP, GTP and cordycepin triphosphate requires removal of the 3′ 2′ O Methyl GMP from G21 by the RNA polymerase followed by elongation to position A78 yielding the Step 2 RNA, FIG. 1. The T9 termination signal lies within the A-less cassette, spanning position +29 to +37 in the G21(TER29)A78 template. Various transcripts can be produced from the Ter29 template as shown in FIG. 1. In the Ter59 template (not shown), the T9 termination signal lies within the A-less cassette, spanning position +59 to +67. The biotinylated 324 bp DNA template was amplified by PCR employing a 5′ biotinylated downstream primer and isolated by preparative agarose gel electrophoresis. The purified DNA fragment was then immobilized to streptavidin-coated magnetic beads (Dynabeads™ M280; Dynal™) according to manufacturer's instructions.

The bead-bound (B) template (about 100 finoles) was first incubated with 4 μl of C50 or WT virus-infected cell extracts, in the presence of 1 mM ATP, 10 μCi α³²P-CTP (800 Ci/mmole), 0.1 mM UTP and 0.625 mM 3′OMeGTP to synthesize the G21 transcript. The ternary complex was isolated, washed twice with 0.02 to 0.5 ml of transcription salts, resuspended and incubated in the presence or absence of VTF that was preincubated in the presence or absence of oligonucleotide for 10 minutes on ice, prior to incubation with the ternary complexes. Termination was then assessed after elongation of the ternary complex in the presence of 1 mM UTP, 1 mM GTP, 1 mM CTP and 1 mM ATP. RNA products were separated by gel electrophoresis, observed by autoradiography and quantified by densitometry of the exposed film. Termination efficiency was calculated as the molar ratio of terminated RNA to the sum of read through and terminated RNA.

As shown in FIG. 1, elongation of the G21 RNA in the presence of all 4 NTPs to the end of the template yields a read through transcript of about 177 bases in length. Normal termination of transcription yields a termination product of about 70 bases in length. Greater than 90 % of the ternary complexes isolated in the first transcription reaction were routinely elongated in the second RNA synthesis reaction.

To assess transcript release from bead bound ternary complexes, the ternary complex was isolated, washed, resuspended and transcript release from the paused ternary complex was assessed. The bound transcript was separated from the free using a magnet, separated by gel electrophoresis and analyzed as described above.

An RNA oligonucleotide, the U5NU-22mer wherein N was U, has a sequence identical to the nascent transcript synthesized from the Ter 29 template from base 21 to 42.

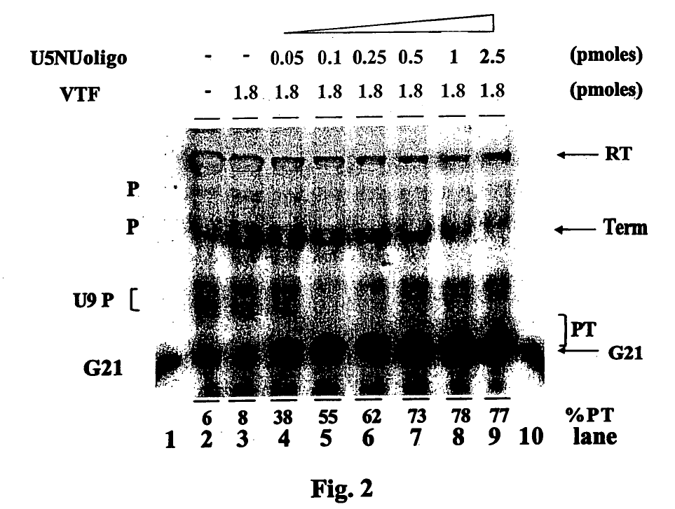

The effect of U5NU-22mer on transcription termination is shown in FIG. 2. The major transcript produced in the absence of added oligonucleotide (lane 2) is the full length read through transcript (RT). Minor shorter products (P), including a collection of pause products at the T9 sequence (U9P), and the unelongated G21 RNA (lanes 1,10) can be identified.

Minimal termination (Term) can be seen, lane 2, probably due to the low level of endogenous VTF. When VTF is added, lane 3, there is a decrease in RT and an increase in Term, as expected when transcription is conducted with a wild type virus infected cell extract. In the presence of VTF plus increasing concentrations of the U5NU-22mer oligonucleotide (lanes 4 to 9) there was a decrease in Term reflecting an inhibition of transcription termination at the normal site. Rather than finding a corresponding increase in RT, an unexpected and dramatic increase in the level of short transcripts (PT) was observed. The concentration of the U5NU-22mer oligonucleotide required for half maximal PT production was about 13 nM. Thus, the addition of an oligonucleotide of the present invention comprising the U5NU sequence stimulates the production of premature transcripts. Additionally, similar results were obtained with a U5NU oligonucleotide-22mer wherein the N was adenine (data not shown).

### EXAMPLE 2

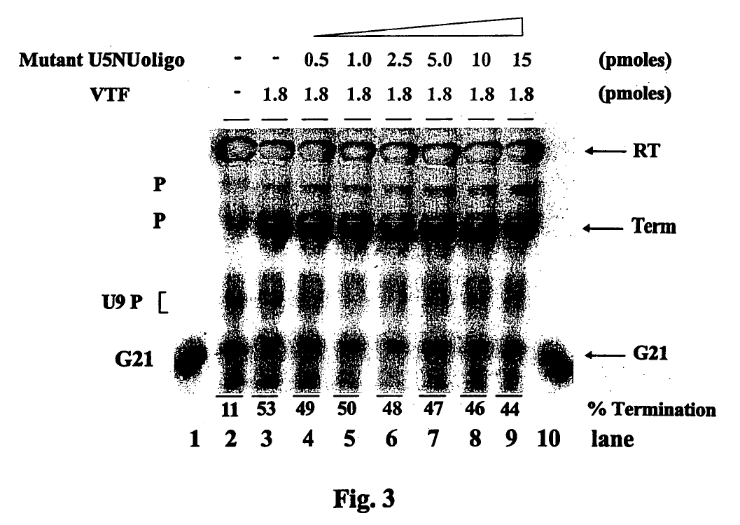

This example demonstrates that the U5NU sequence is required for the premature termination of transcription. To illustrate the requirement of the U5NU sequence, an oligonucleotide which does not have the U5NU sequence (mutant-22mer) was used. In the mutant-22mer, selected U residues were changed to A at positions 8, 9, 12, and 15 to yield an altered termination signal such that the U5NU sequence became UUUUAUU (SEQ ID NO:20), which generates a sequence that is inactive in transcription termination. As shown in FIG. 3, incubation with the mutant-22mer did not result in an increase in premature transcripts. This demonstrates the requirement for the U5NU sequence for stimulation of premature transcription termination.

### EXAMPLE 3

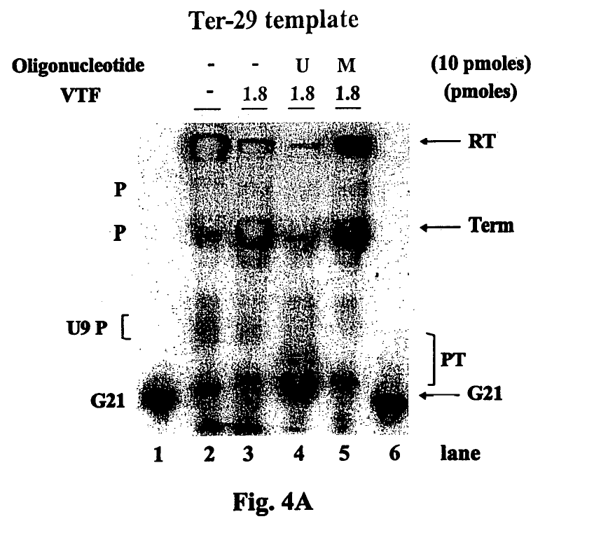

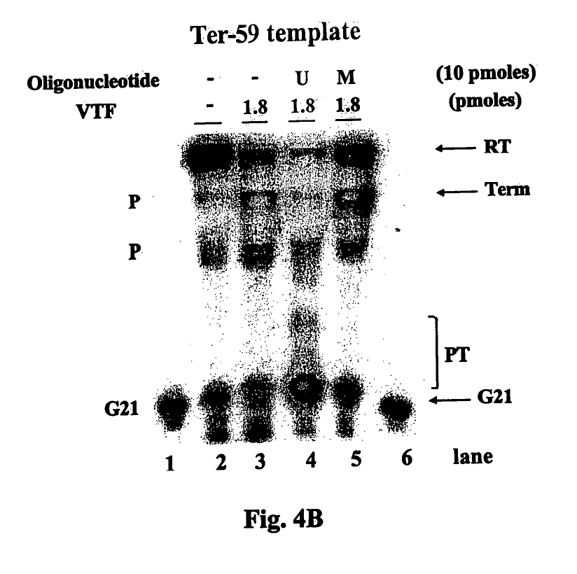

To determine whether signal dependent stimulation of premature transcription termination was template dependent, the effect of oligonucleotides on transcription termination was evaluated using two different templates, the Ter 29 and Ter 59 templates. Ter 29 was described in Example 1. G21(TER59)A78 (Ter 59) was the other template used. While Ter 29 has the termination signal start at position 29, in Ter 59 the termination signal start is at position 59. Transcription reactions were carried out as described in Example 1. The results are shown in FIG. 4A and FIG. 4B for the Ter 29 and the Ter 59 templates respectively. The normal termination product produced from the Ter-59 template is longer than that synthesized from Ter 29. Addition of the U5NU-22mer but not the mutant-22mer results in a decrease in the normal termination product and an increase in the synthesis of the prematurely terminated RNA from both templates (lanes 4,5). Each template yielded similarly sized premature termination products demonstrating that the ability of the oligonucleotides to generate premature termination products is not template dependent. Further, the premature termination effected by the U5NU-22mer utilizes the highly conserved transcription termination machinery of poxviruses as evidenced by enhanced premature termination in the presence of VTF.

### EXAMPLE 4

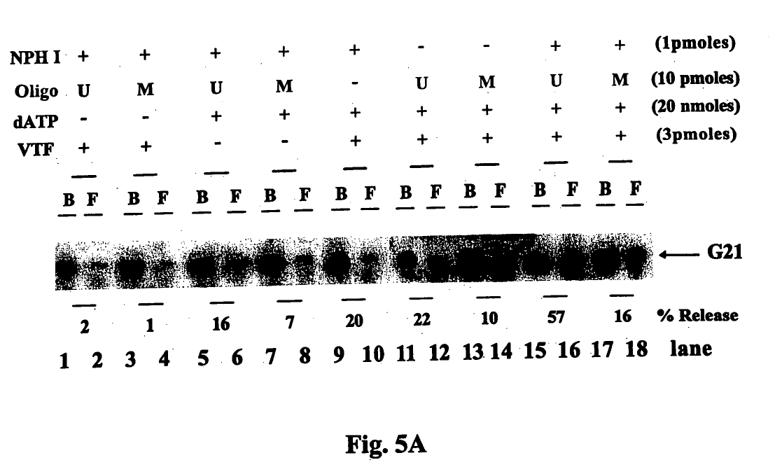

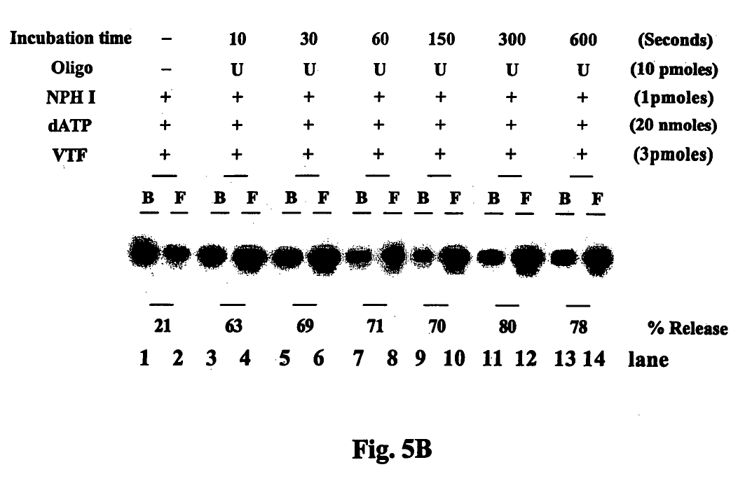

This example demonstrates that the factors known to be required during normal termination enhance premature termination. A temperature sensitive (“ts”) mutant virus, tsC50, was propagated in BSC40 African green monkey cells at the permissive temperature of 31° C. The tsC50 virus harbors a ts mutation which encodes a mutant NPHI that is active only at the permissive temperature. When viral transcription extracts are prepared from tsC50 grown at the non-permissive temperature, exogenous NPHI must be added for maximum transcription termination. Cell infection was carried out as follows. A549 cells were infected with tsC50 mutant virus at a multiplicity of infection (m.o.i.) of 15, at the nonpermissive temperature of 40° C. or 31 ° C. After 24 hours, the medium was removed and replaced with 40° C. medium containing 100 μg/ml of cycloheximide. After a further 24 hours at 40° C, cells were washed and treated with 250 μg/ml lysolecithin and extracts prepared by standard methods. Ternary complexes were constructed using a tsC50 mutant virus infected cell extract and further incubations of the isolated, washed ternary complexes were conducted in the absence or presence of NPH I, VTF and DATP, and in the absence or presence of the wild type U5NU-22mer wherein U was N, or the mutant-22mer wherein U was N. After further incubation, bead bound and free G21 RNA was quantified after gel electrophoresis to evaluate the U5NU oligonucleotide dependent transcript release. The results are shown in FIG. 5A, wherein U5NU-22mer is shown as “U” and the mutant-22mer is shown as “M”. When dATP is omitted (lanes 1 to 4, FIG. 5A) there is little release of the premature termination transcript, G21 RNA due to the lack of an energy source. If VTF is omitted (lanes 5 to 8) there is a slight stimulation of release (16%) when the mutant-22mer is added, probably due to the low level of endogenous VTF in this assay. The mutant-22mer oligonucleotide has a much lower effect on release (lanes 7,8). If NPH I is omitted (lanes 11 to 14) a low level of transcript release is observed which is higher (22%) when the U5NU-22mer oligonucleotide is added rather than the mutant-22mer (10%). If oligonucleotide is not added (lanes 9, 10) only 20% release is observed. However, in the presence of VTF, NPHI, and DATP a high level (57%) of G21 RNA release can be observed in the presence of the U5NU-22mer oligonucleotide (lanes 15,16). Only background release is observed when the mutant-22mer is added (lanes 17, 18).

As shown in FIG. 5B, the release of G21 RNA from the isolated bead bound temary complex is rapid since release is observed within 10 seconds after initiating -the reaction. These results demonstrate that the oligonucleotides of the present invention comprising the U5NU sequence stimulate rapid release of the transcript from the viral polymerase and does not appear to require ongoing transcription.

### EXAMPLE 5

This Example illustrates additional sequence and structural features of the oligonucleotides of the present invention by assessing the ability of various oligonucleotides to produce premature transcripts. Transcription was carried out as described in Example 1.

Both SEQ ID NO:8 (“dU5NdU” hereinafter) and SEQ ID NO:9 (“BrdU5NdU” hereinafter) are 22mer RNA/DNA chimeras that are identical to the U5NU-22 mer, except for the substitution of the U5NU sequence with either dU5NdU, in which all the U's are deoxyribouracils, or BrdU5NU, in which the all U's are deoxyuracils brominated at the 5 position. Two ssDNA oligonucleotides, SEQ ID NO: 10 (“T5NT” hereinafter) and SEQ ID NO:11 (“mutant-T5NT” hereinafter), represent the DNA equivalent of the U5NU-22mer and mutant-22mer, respectively. Each oligonucleotide was analyzed via their affect on transcription of the bead bound templates as described in Example 1.

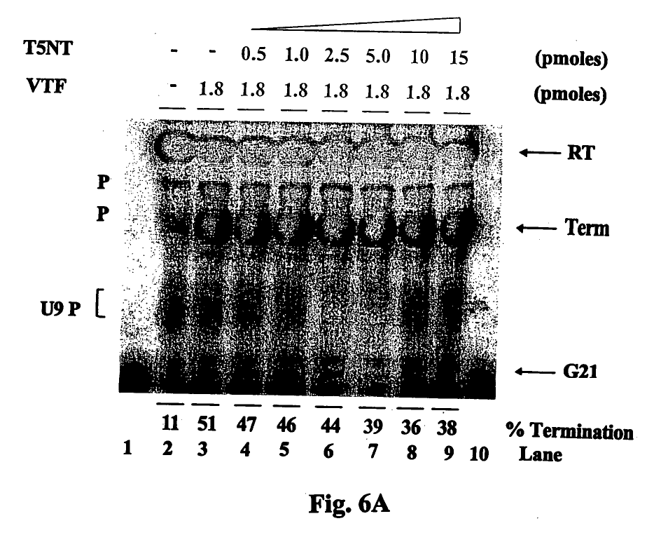

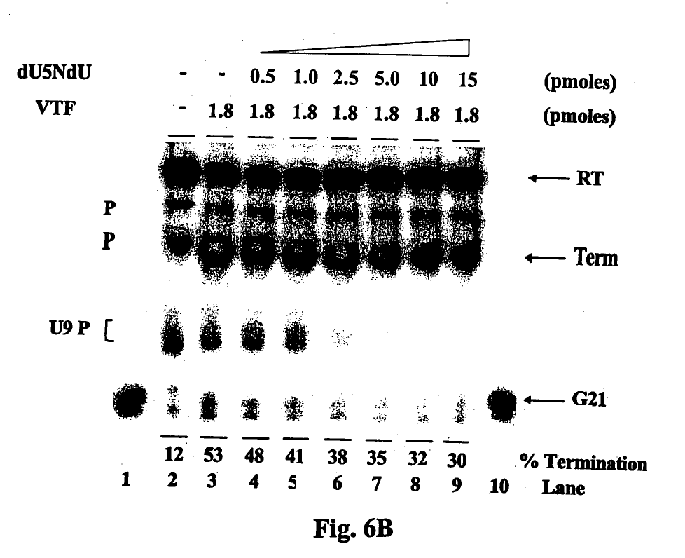

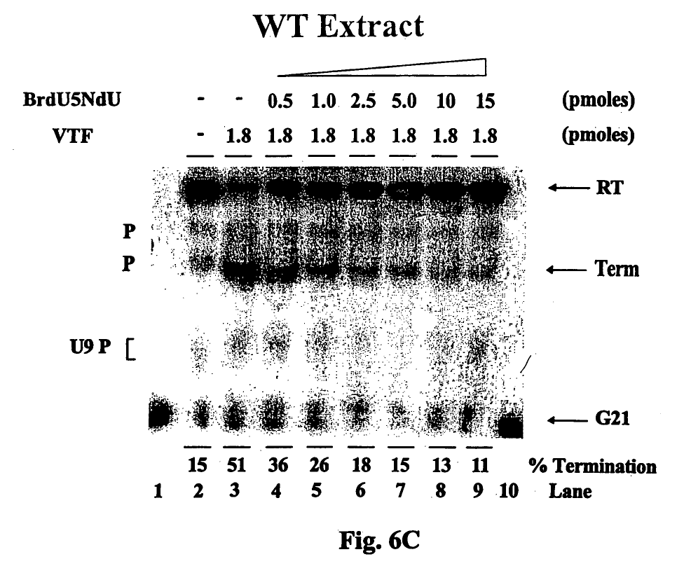

The results are shown in FIG. 6A. Addition of increasing concentrations of T5NT failed to stimulate premature termination and exhibited a similar general inhibition of normal termination at high concentration (FIG. 6A). Similar results were obtained using mutant-T5N-T sequence.

In FIG. 6B, the chimeric RNA/DNA oligonucleotide dU5NdU failed to stimulate premature termination, demonstrating the requirement for ribose in the U5NU sequence.

In order to evaluate the effect of substitution of U with BrdU in the U5NU sequence, the effect of adding increasing concentrations of the BrdU9 containing chimeric oligonucleotide BrdU5NdU were tested on transcription termination (FIG. 6C). Addition of BrdU5NdU significantly decreased normal transcription termination, exhibiting a corresponding increase in the read through product. However, BrdU5NdU did not stimulate premature termination. These results demonstrate that the oligonucleotides of the present invention require a ribonucleotide portion containing the U5NU sequence to stimulate premature transcription termination.

### EXAMPLE 6

This Example demonstrates that oligonucleotides longer than 22-mers can also effect premature termination of transcription. The in vitro synthesis of two 36-mer oligonucleotides, SEQ ID NO:5 (“pGEM-U5NU-36mer” hereinafter) wherein N was U, and SEQ ID NO:6, (“pGEM-BrU5NU-36mer” hereinafter), was carried out as follows:

The transcription template plasmid was constructed whereby an oligonucleotide having a sequence of aattgggccggctttttttttgcgttg (SEQ ID NO:7) was synthesized and annealed to an oligonucleotide of the complimentary sequence yielding a double-stranded oligonucleotide containing identical 4-base 5′ overhangs. This fragment was ligated into the EcoRI site of pGEM3Zf(+) and the orientation was determined by sequencing the DNA. The downstream end retained the EcoRI recognition site so that cleavage with EcoRI yielded a linear template used in run off transcription. One ml transcription reactions contained 200 μg of linearized plasmid DNA, 10 mM DTT, 1 mM ATP, CTP, GTP and UTP (or BrUTP in the case of pGEM-BrU5NU-36mer), and 500 units of T7 RNA polymerase. In order to facilitate the detection of RNA during purification, 2.5 μCi [α-³²P]-CTP was included in each reaction. Transcription reactions were carried out for 2 hrs at 37° C. The reactions were quenched, and protein was extracted by adding an equal volume of 24:24:1 phenol-STE:chloroform:isoamyl alcohol. The RNA was precipitated with 2.5 M ammonium acetate and three volumes of 95% ethanol. Transcription products were separated by electrophoresis on 10% acrylanide, 8 M urea gels and excised from the wet gels using an autoradiograph as a guide. RNA was eluted from the gel using conventional techniques, precipitated with 0.3 M sodium acetate and three volumes of ethanol, dried and resuspended in 250 μl of H20. Transcription of the plasmid linearized at the EcoRI and in the presence of UTP site yielded the pGEM-U5NU-36mer oligonucleotide, while transcription carried out in the presence BrUTP resulted in the pGEM-BrU5NU-36mer.

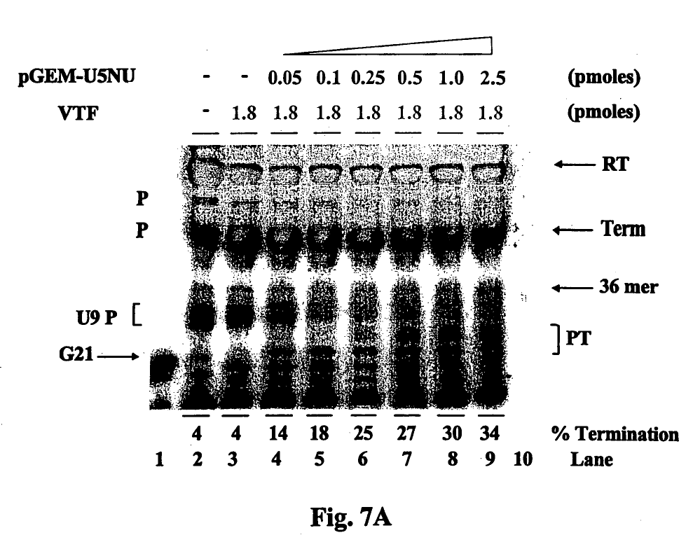

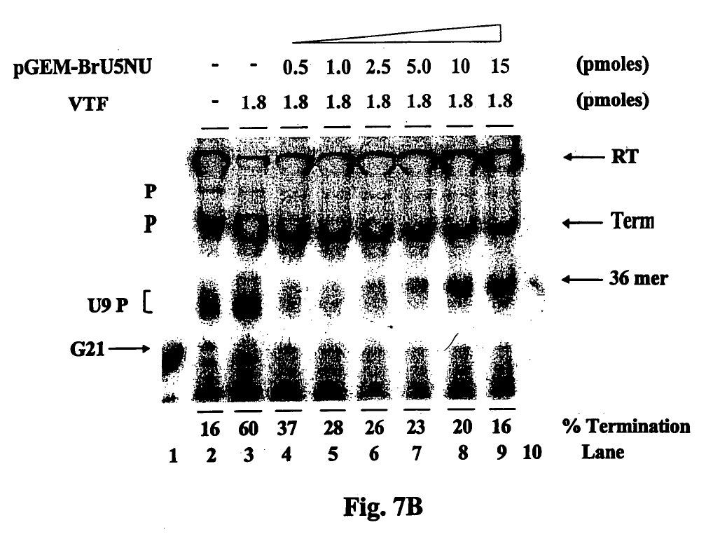

Similar to the results obtained with the U5NU-22mer oligonucleotide, addition of pGEM-U5NU-36-mer stimulated premature transcription termination in a concentration dependent manner (FIG. 7A, lanes 4-9). In contrast, addition of pGEM-BrU5NU-36-mer, in which the uracils in the U5NU sequence were brominated, significantly decreased normal transcription termination, exhibiting a corresponding increase in the read-through product, yet failed to stimulate premature transcription termination (FIG. 7B). These results demonstrate a requirement for unbrominated uracil for the U5NU-based stimulation of premature termination and also demonstrate that premature termination can be effected by oligonucleotides longer than 22-mers.

### EXAMPLE 7

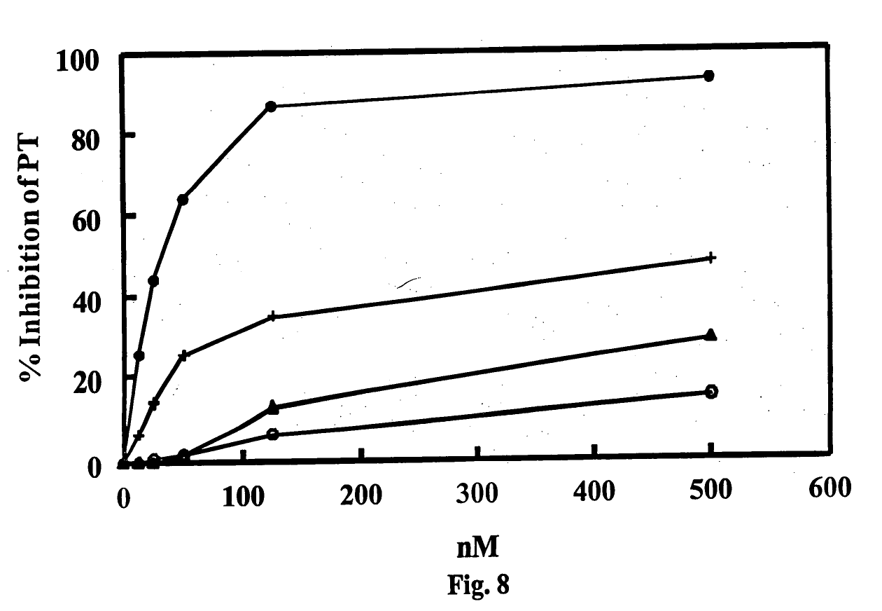

This example demonstrates that certain oligonucleotides with BrdU9 can act as competitive inhibitors of U5NU and thus can inhibit the formation of premature termination transcripts by oligonucleotides comprising the U5NU sequence. Transcription was carried out as described in Example 1 in the presence of various concentrations of competitive inhibitors. The results are shown in FIG. 8 for the effect of BrdU5NdU ( ); mutant-22mer (▴);T5NT (O); and dU5NdU(+) on premature transcription termination by U5NU-22mer, wherein N was U. Addition of BrdU5NdU prevented premature termination in a concentration dependent manner. Furthermore, inhibition of normal termination was observed at high BrdU5NdU concentrations, resulting in increased read through RNA synthesis (data not shown). In contrast to BrdU5NdU, the other oligonucleotides containing U5NU modifications did not show significant inhibition of the U5NU stimulation of premature termination. These data sugest that BrdU5NdU was able to serve as a competitive inhibitor of the U5NU-22mer stimulated release of the G21 transcript (data not shown).

These results demonstrate that although BrdU5NdU fails to stimulate both premature termination and transcript release, it can compete with U5NU in a manner suggesting competition for a factor present in poxvirus infections necessary for stimulation of premature termination of transcription. Further, this Example again demonstrates the requirement for unmodified ribose in the U5NU sequence to effect stimulation of premature termination of transcription.

### EXAMPLE 8

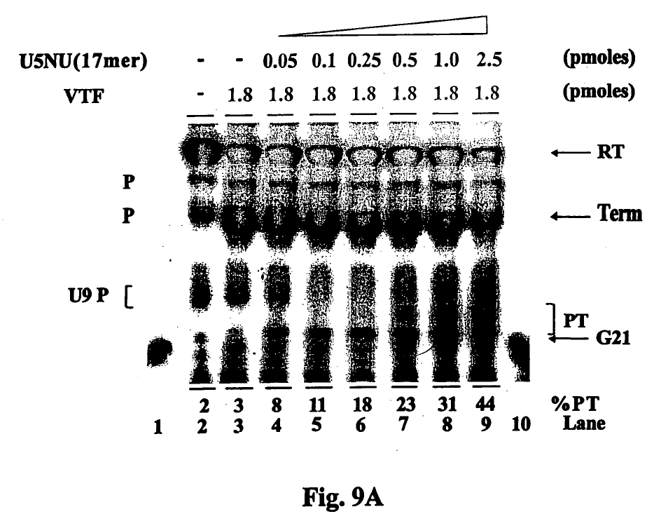

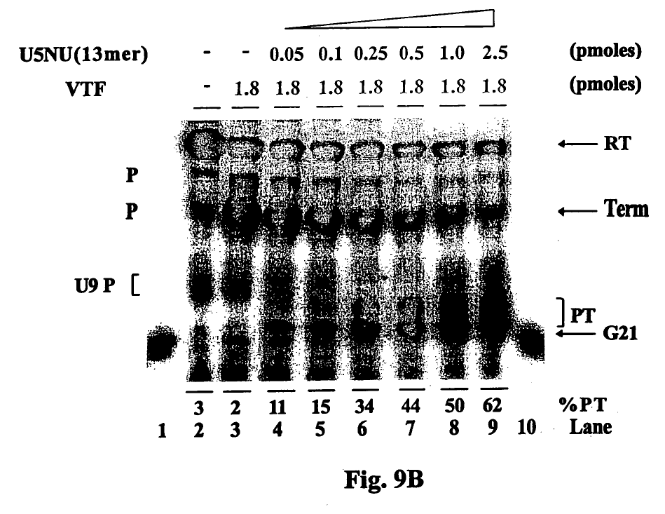

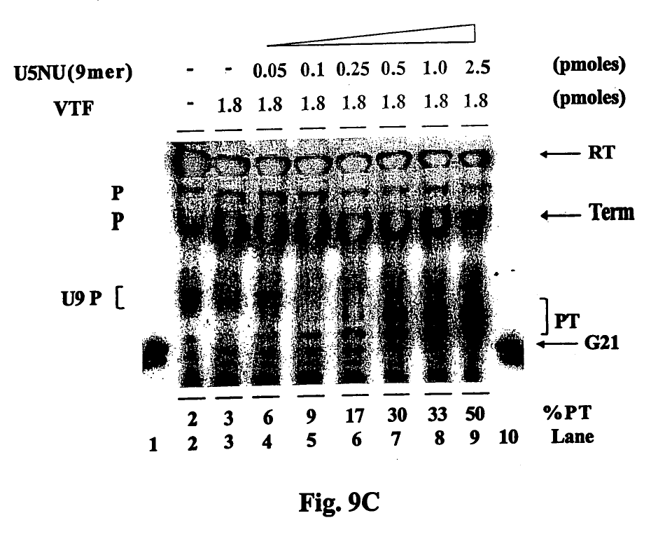

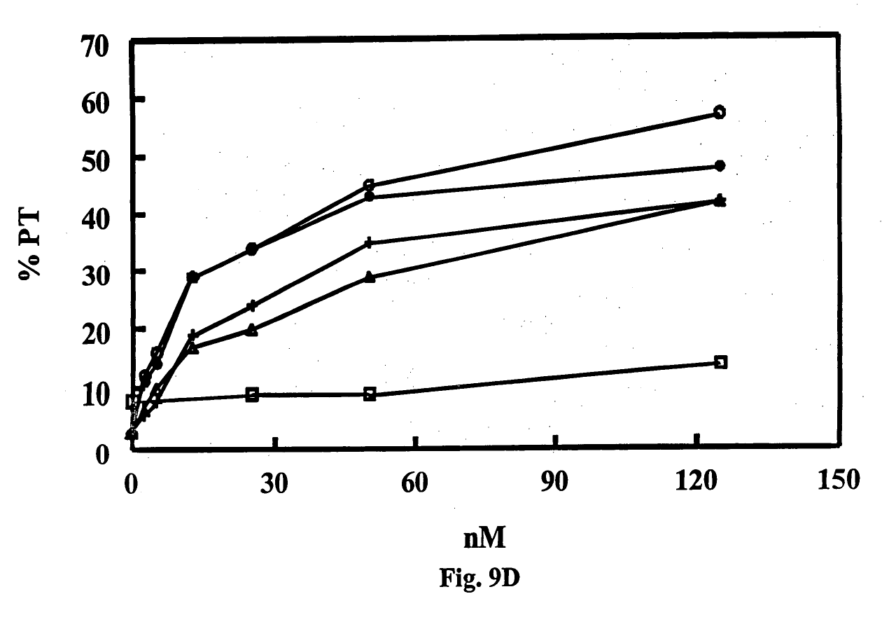

This example demonstrates that oligonucleotides of different lengths can be useful for premature termination of transcription. In order to illustrate this embodiment, U5NU containing oligonucleotides in lengths of 17 nucleotides (U5NU 17-mer; SEQ ID NO:12), wherein N is U, (FIG. 9A); 13 nucleotides (U5NU-13-mer; SEQ ID NO:13),wherein N is U, (FIG. 9B); and 9 nucleotides, (U5NU-9-mer; SEQ ID NO:14), wherein N is U (FIG. 9C), stimulated premature termination. In contrast, both a U5NU-7-mer (SEQ ID NO: 15) wherein N is U, and U5AU-7-mer (SEQ ID NO:16) were unable to support premature transcription termination (data not shown). FIG. 9D is a graphical representation of the average of multiple analyses of simulation of premature termination by oligonucleotides of different lengths comprising the U5NU sequence. Data are shown for U5NU-22-mer (●);U5NU-17-mer (Δ);U5NU-13-mer (O);U5NU-9-mer (+); and U5NU-7-mer (□). The concentration of each oligonucleotide required for half maximal stimulation of premature termination, A_{0.5}, was calculated from a reciprocal plot of the average data and results in a range of A_{0.5} 13 to 33 nM oligonucleotide concentrations.

### EXAMPLE 9

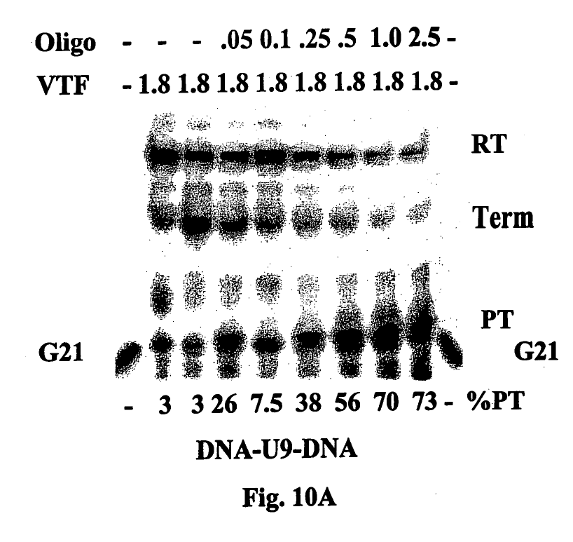

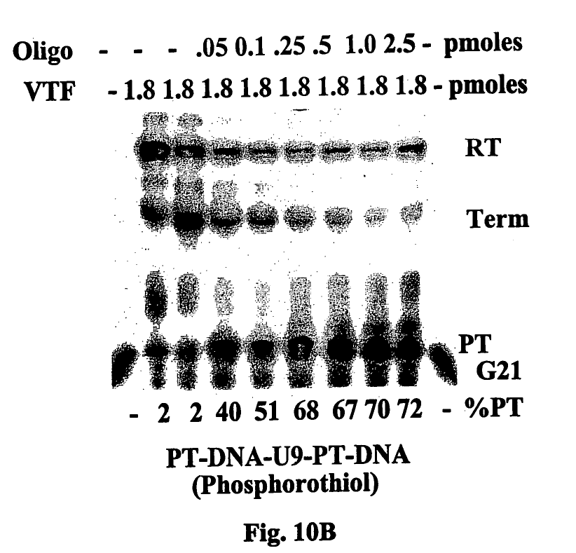

This example demonstrates that the oligonucleotides of the present invention can be chimeric oligonucleotides. The flanking regions of the oligonucleotides may comprise nucleotides, deoxyriboucleotides or modifications thereof. As an illustration, two chimeric oligonucleotides were tested for their ability to generate premature transcription termination products. The two chimeric oligonucleotides tested were DNA-U9-DNA (SEQ ID NO:17) in FIG. 10A and PT-DNA-U9-PT-DNA (SEQ ID NO:19) in FIG. 10B. The results shown in FIG. 10A and 10B demonstrate that the flanking sequences can be modified in the phosphodiester bond and the 2′ position of the sugar without affecting termination function. FIG. 10A demonstrates that a chimeric 22-mer with unmodified U5NU flanked by normal DNA sequences can stimulate premature transcription termination. FIG. 10B similarly demonstrates that a chimeric 22-mer with unmodified U5NU flanked by regions of DNA with phosphorthioldiester bonds can also stimulate premature transcription termination.

These results demonstrate that chimeric oligonucleotides that comprise a U5NU sequence flanked on at least on side by at least one nucleotides, which can be a ribonucleotide, deoxyribonucleotides or modifications thereof retain their ability to stimulate premature transcription termination.

### EXAMPLE 10

This embodiment demonstrates that the effects of the oligonucleotides of the present invention are also seen on viral core RNA synthesis.

Propagation of the wild type (WT) vaccinia virus strain WR and virus titers determinations were performed as in Example 1. Purification of vaccinia virions was carried out as follows. Briefly, vaccinia virus was grown in BSC40 cells and purified from the cytoplasmic fraction by sedimentation through a cushion of 36% (w/v) sucrose and two successive 25 to 40% (w/v) sucrose gradient sedimentations. The virion band was separated, diluted and virions were pelleted by centrifugation. The virions were then resuspended in 10 mM Tris-HCl, pH 7.5.

Transcription using permeabilized virions performed as follows: The purified virions were first preincubated with 10 mM dithiothreitol (DTT) and 0.05% Nonidet P-40 (NP-40) for 3 minutes at 37° C. followed by the addition of the remaining reagents, and incubation was continued for 20 minutes at 37° C. Typically, 40 μl reaction mixtures containing 60 mM Tris-HCl, pH 8.0, 10 mM MgCl₂, 10 mM dithiothreitol, 5 mM ATP, 1 mM UTP, 1 mM GTP, 0.6 μM CTP, 6.6 μCi α³²P-CTP (800 Ci/mmole), 10 μM AdoMet, 0.05% Nonidet P-40, and purified vaccinia virions (0.1 A₂₆₀/ml) were incubated at 37° C. for 20 minutes. Reactions were quenched with 160 μl of 50 mM Tris-HCl, pH 8.0, 10 mM EDTA, 10 mM dithiothreitol and 0.05% Nonidet P-40. Virus cores were separated from released RNA by centrifugation for 5 minutes at 13,000 rpm in a microcentrifuge. Samples of released RNA were made 0.1% in sodium dodecyl sulfate, extracted with phenol:CHCl₃ and precipitated with ammonium acetate and isopropanol. Virus cores were treated with 20 μg proteinase K for 2 hours at 37° C., then were extracted and precipitated as described for released RNA. Core-bound and released RNAs were analyzed by electrophoresis through 1% agarose gels containing 2.2 M formaldehyde. Gels were dried and labeled RNAs were visualized by autoradiography. Lanes containing co-electrophoresed unlabeled RNA size standards were cut out, and RNA bands were located by staining with ethidium bromide.

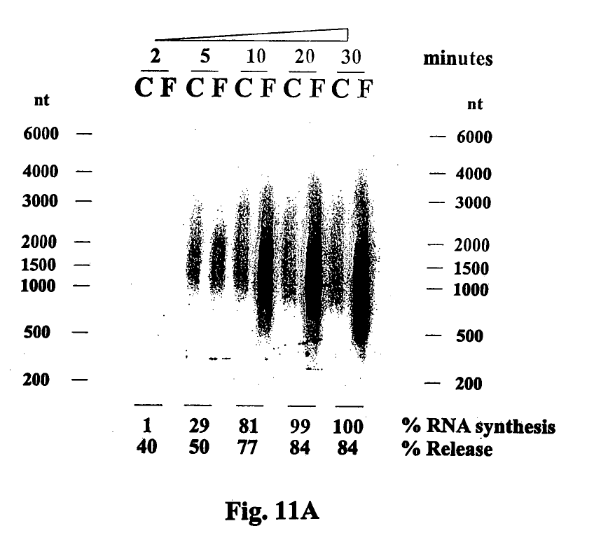

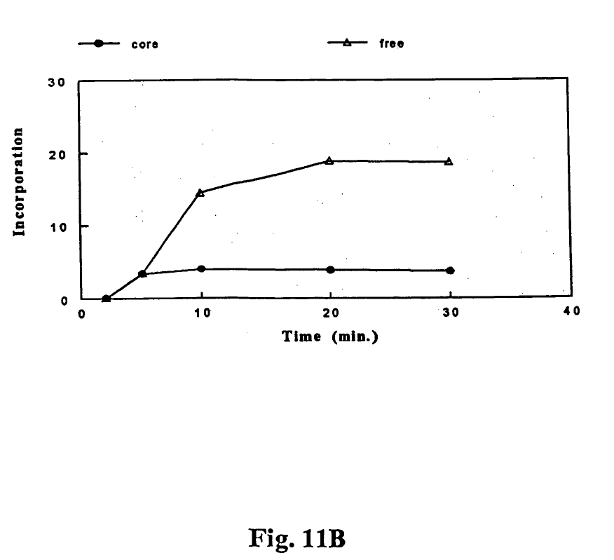

The time course of core RNA synthesis in viral cores and release of transcripts was evaluated and the results are shown in FIG. 11A. Analysis of core-associated (C) and free (F) RNAs showed that while the core-retained RNA rapidly achieved a steady state level, free RNA accumulated in a time dependent manner. Maximum RNA synthesis and release (84%) of the nascent transcript was achieved after 20 minutes (FIG. 11B).

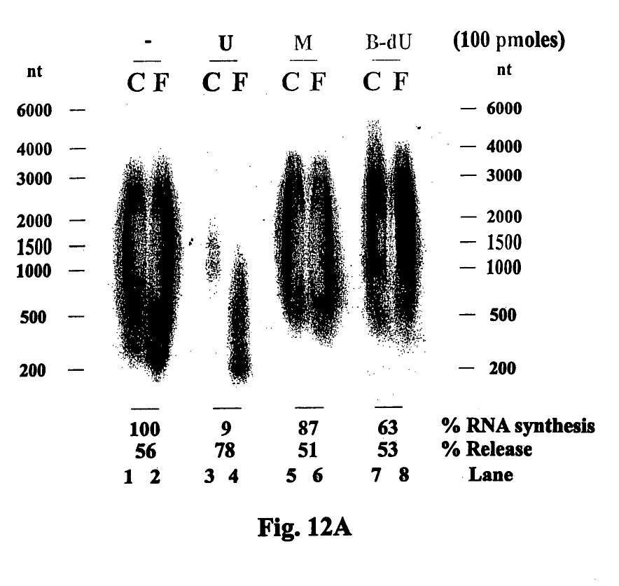

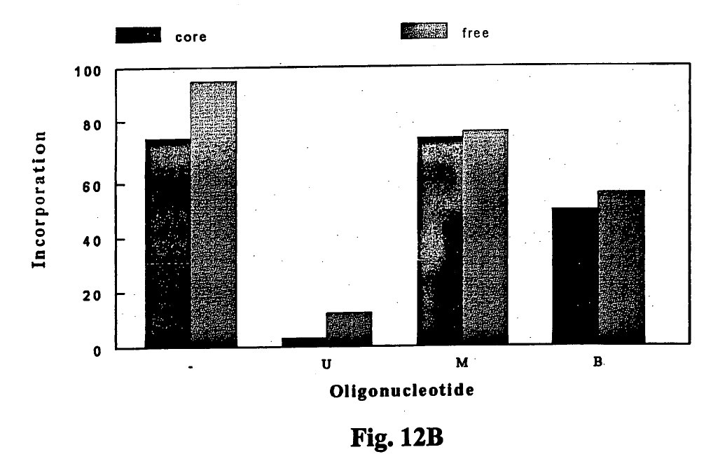

In order to evaluate the effect of the different oligonucleotides on core RNA synthesis, transcription was carried out in the absence or presence of 100 pmoles of U5NU-22mer (U), the mutant-22mer (M), or BrdU5NdU (B-dU) (FIG. 12A). In the absence of added oligonucleotide, both core-bound and free RNA are synthesized. Addition of the U5NU-22mer resulted in a dramatic inhibition of core RNA synthesis with the average size of the product RNA being shorter than the untreated RNA (FIG. 12A, lanes 1-4). In contrast, the mutant-22mer oligonucleotide failed to cause any significant inhibition of RNA synthesis, again demonstrating the requirement for the U5NU sequence (FIG. 12A, lanes 5-6). The BrdU5NdU oligonucleotide exhibited a slight inhibition of core RNA synthesis (FIG. 12A, lanes 7-8). A graphical representation of the results depicted in FIG. 12A is shown in FIG. 12B. The results obtained with viral cores are consistent with the results seen with extracts in Examples 2-9.

### EXAMPLE 11

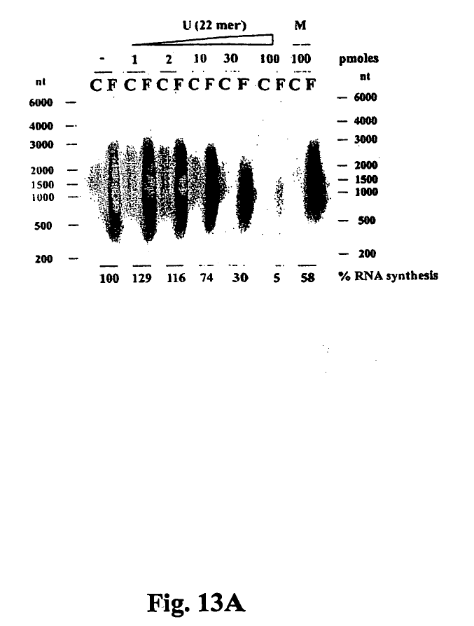

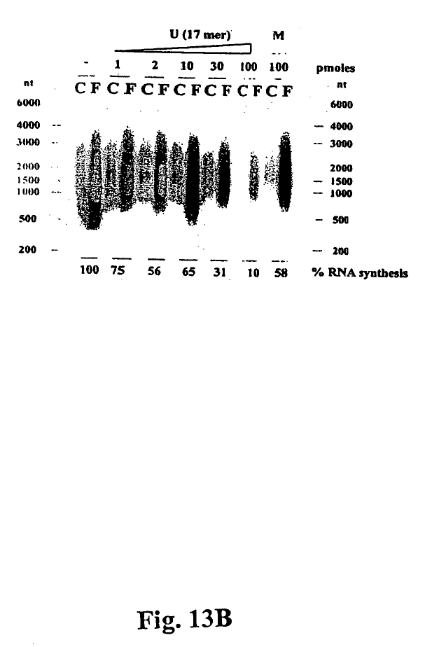

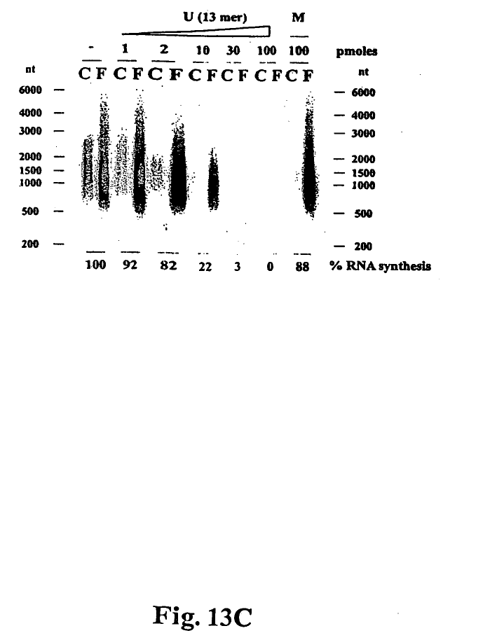

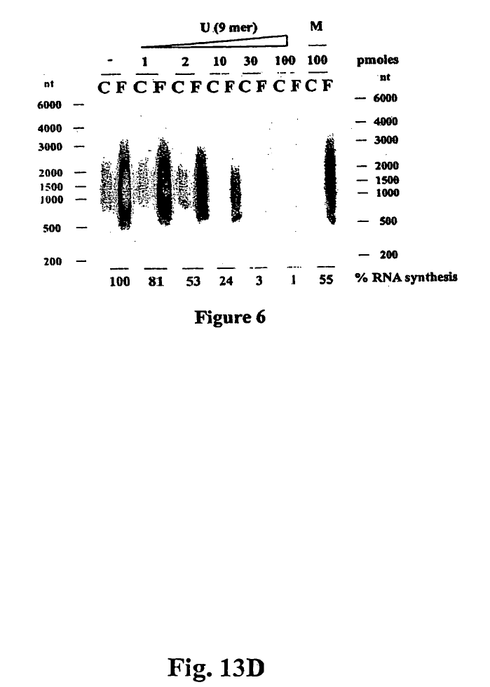

In Example 8, it was demonstrated that oligonucleotides of different lengths could generate premature transcription termination products. In this example, experiments were carried out to determine if effects of oligonucleotides of different lengths could also be observed on the inhibition of core RNA synthesis. Transcription was carried out as described in Example 10 in the presence or absence of increasing concentrations of the oligonucleotides of different lengths comprising the U5NU sequences (the U5NU-22mer, U5NU-17-mer, U5NU-13-mer, U5NU-9-mer or U5NU-7-mer). Generation of premature termination products were observed for U5NU-22mer, 17-mer, 13-mer and 9-mer (FIGS. 13A, 13B, 13C and 13D, respectively). In contrast, neither the U5NU-7-mer nor U5AU were able to significantly inhibit core RNA synthesis (data not shown). The concentration of each oligonucleotide required for half maximal inhibition of core RNA synthesis, 10.5, was calculated from a reciprocal plot of the average data. These results demonstrate that shorter oligonucleotides comprising the U5NU sequence are more effective inhibitors of core RNA synthesis than the U5NU-22mer.

### EXAMPLE 12

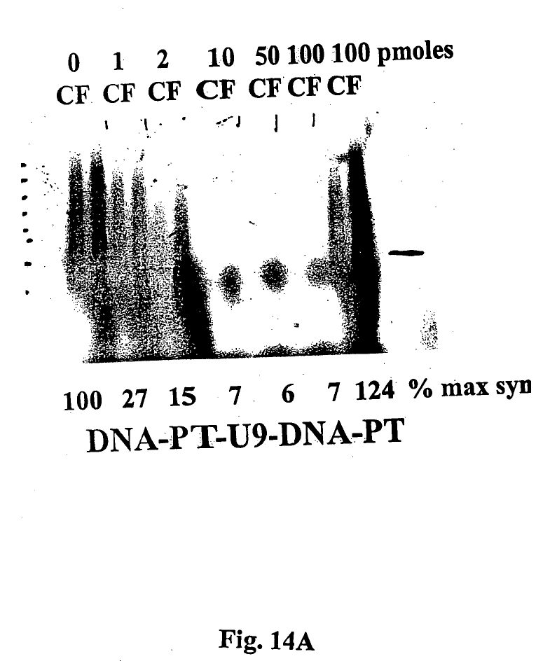

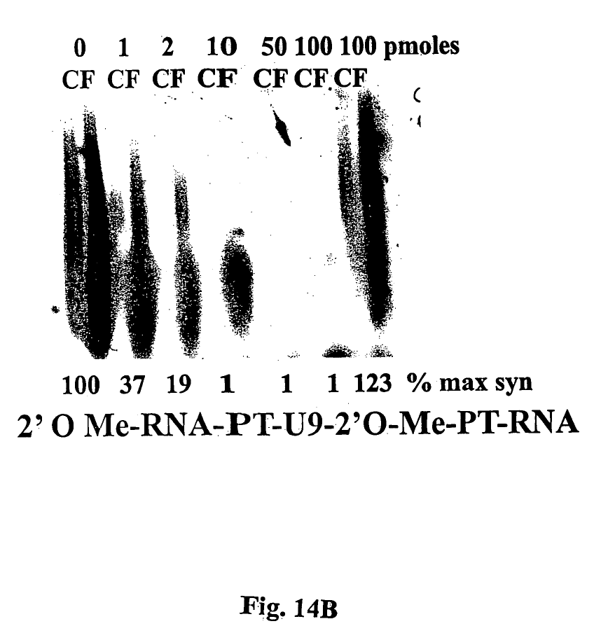

This example demonstrates the ability of chimeric oligonucleotides to inhibit core mRNA synthesis. As an illustration, DNA-PT-U9-DNA-PT and 2′O-Me-RNA-PT-U9-2′O-Me-PT-RNA (SEQ ID NO:18) were used. Core RNA synthesis was determined as described in Example 10. The results are shown in FIG. 14A for DNA-PT-U9-DNA-PT and demonstrate that this oligonucleotide, in which the flanking regions were deoxyribonucleotides modified in the phosphodiester bond, was able to inhibit RNA synthesis and stimulate premature transcription termination.

FIG. 14B demonstrates that the chimeric oligonucleotide, 2′O-methyl-RNA-phophorothiol-U9-2′O-methyl-RNA-phophorothiol (SEQ ID NO: 18) oligonucleotide is also able to generate premature termination products in viral cores.

These results demonstrate that chimeric oligonucleotides comprising a U5NU sequence flanked on at least one side by at least one nucleotide which is not a ribonucleotide retain their ability to stimulate premature transcription termination. Further, this demonstrates that the chimeric oligonucleotides of the present invention can gain access to viral cores and cause pretermination of transcription therein.

### EXAMPLE 13

This example demonstrates that the oliognucleotides of the present invention are effective against poxviruses other than vaccinia virus. As an illustration, the ability of the U5NU-22mer and the DNA-U9′-DNA chimeric oligonucleotide to stimulate premature transcription termination in ectromelia (mousepox) virus was tested. Purification of mousepox virions, transcription using permeabilized virions and premature transcription termination assays were performed in a manner similar to that described in Example 10. The results indicate that the oligonucleotides of the present invention stimulate premature termination of transcription in mouse pox in a manner similar to that observed for vaccinia, and thus can inhibit replication of poxviruses other than vaccinia accordingly.

Although the present invention has been demonstrated by the examples presented above, it will be appreciated by those skilled in the art that routine modifications are possible without departing from the scope of the invention as described in the specification and the claims.

References

1. Moss, B. (ed) (2001) Poxviridae: the viruses and their replication Vol. 2. Virology. Edited by Knipe, D. M., Howley, P. M., Griffin, D. E., Martin, M. A., Lamb, R. A., Roizman, B., and Strauss, S. E. 2 vols., Lippincott-Raven, Philadelphia
2. Moss, B., Ahn, B. Y., Amegadzie, B., Gershon, P. D., and Keck, J. G. (1991) Journal of Biological Chemistry 266, 1355-1358
3. Baroudy, B. M., and Moss, B. (1980) Journal of Biological Chemistry 255, 4372-4380
4. Ahn, B. Y., and Moss, B. (1992) Proceedings of the National Academy of Sciences of the United States of America 89, 3536-3540
5. Kane, E., and Shuman, S. (1992) J. Virol. 66, 5752-5762
6. Wei, C. M., and Moss, B. (1974) Proceedings of the National Academy of Sciences of the United States of America 71, 3014-3018
7. Kates, J. R., and Beeson, J. (1970) J. Mol. Biol. 50, 19-33
8. Vos, J. C., Sasker, M., and Stunnenberg, H. G. (1991) EMBO Journal 10, 2553-2558
9. Rosales, R., Harris, N., Ahn, B. Y., and Moss, B. (1994) Journal of Biological Chemistry 269, 14260-14267
10. Keck, J. G., Baldick, C. J., Jr., and Moss, B. (1990) Cell 61, 801-809
11. Gunasinghe, S. K., Hubbs, A. E., and Wright, C. F. (1998) J. Biol. Chem. 273, 27524-27530
12. Rosales, R., Sutter, G., and Moss, B. (1994) Proceedings of the National Academy of Sciences of the United States of America 91, 3794-3798
13. Wright, C., Oswald, B., and Dellis, S. (2001) J.Biol. Chem. 276,40680-40686
14. Rohrrmann, G., Yuen, L., and Moss, B. (1986) Cell 46, 1029-1035
15. Mohamed, M. R., Christen, L., and Niles, E. G. (2002) Virology 299, 142-153
16. Shuman, S., Broyles, S. S., and Moss, B. (1987) Journal of Biological Chemistry 262, 12372-12380
17. Morgan, J. R., Cohen, L. K., and Roberts, B. E. (1984) J. Virol. 52
18. Niles, E. G., Lee-Chen, G. J., Shuman, S., Moss, B., and Broyles, S. S. (1989) Virology 172, 513-522
19. Ensinger, M. J., Martin, S. A., Paoletti, E., and Moss, B. (1975) Proceedings of the National Academy of Sciences of the United States of America 72, 2525-2529
20. Deng, L., Shuman, and S. (1998) Genes and Development 12, 538-546
21. Christen, L. M., Sanders, M., Wiler, C., and Niles, E. G. (1998) Virology 245, 360-371
22. Mohamed, M. R., and Niles, E. G. (2000) J. Biol. Chem. 275, 25798-25804
23. Mohamed, M. R., and Niles, E. G. (2001) J. Biol. Chem. 276, 20758-20765
24. Piacente, S. C., Christen, L.M., Mohamed, M.R. and Niles, E.G. (2002) Virology, in press.
25. Shuman, S., and Moss, B. (1989) Journal of Biological Chemistry 264, 21356-21360
26. Deng, L., Hagler, J., and Shuman, S. (1996) Journal of Biological Chemistry 271, 19556-19562

1. An oligonucleotide of between 8 to about 40 nucleotides comprising:
a) an oligoribonucleotide portion which has the sequence of SEQ ID NO: 1; and
b) a flanking region at the 5′ end, the 3′ end or both ends of the oligoribonucleotide portion comprising at least one ribonucleotide, deoxyribonucleotide, modified ribonucleotide or modified deoxyribonucleotide, wherein the oligonucleotide is capable of causing premature termination of transcription of poxvirus genes.

2. The oligonucleotide of claim 1, wherein the oligonucleotide is between 9 and 36 nucleotides long.

3. The oligonucleotide of claim 2, wherein the oligonucleotide is between 9 and 22 nucleotides long.

4. The oligonucleotide of claim 3, wherein the oligonucleotide is between 9 and 13 nucleotides long.

5. The oligonucleotide of claim 1, wherein the sequence of the oligonucleotide is selected from the group consisting of SEQ ID NO:3, SEQ ID NO:5, SEQ ID NO: 12, SEQ ID NO:13, SEQ ID NO:14, SEQ ID NO:17, SEQ ID NO:18 and SEQ ID NO:19.

6. The oligonucleotide of claim 1, wherein the sequence of the oligonucleotide is SEQ ID NO: 14.

7. The oligonucleotide of claim 1, wherein the flanking region comprises at least one nucleotide in which the sugar residue is modified at the 2′O position.

8. The oligonucleotide of claim 7, wherein the modification is selected from the group consisting of CH3, CH30, NH2 and CH3CH2NH2.

9. The oligonucleotide of claim 1, wherein the flanking region has inter-nucleoside linkages selected from the group consisting of phosphorothiols, methylphosphonates and phosphoramidites.

10. A composition comprising the oligonucleotide of claim 1.

11. The composition of claim 10, wherein the sequence of the oligonucleotide is selected from the group consisting of SEQ ID NO:3, SEQ ID NO:5, SEQ ID NO:12, SEQ ID NO:13, SEQ ID NO:14, SEQ ID NO:17, SEQ ID NO:18 and SEQ ID NO:19.

12. The composition of claim 10, wherein the composition comprises a pharmaceutically acceptable carrier.

13. A method for generating premature transcription termination products which are characterized as running at about 21 to about 30 bases by gel electrophoresis, from cells infected with a poxvirus comprising the steps of providing to a cell infected with the poxvirus an oligonucleotide of between 8 to about 40 nucleotides comprising:
a) an oligoribonucleotide portion which has the sequence of SEQ ID NO: 1; and
b) a flanking region at the 5′ end, the 3′ end or both ends of the oligoribonucleotide portion comprising at least one ribonucleotide, deoxyribonucleotide, modified ribonucleotide or modified deoxyribonucleotide.

14. The method of claim 13, wherein the sequence of the oligonucleotides is selected from the group consisting of SEQ ID NO:3, SEQ ID NO:5, SEQ ID NO:12, SEQ ID NO:13, SEQ ID NO:14, SEQ ID NO:17, SEQ ID NO:18 and SEQ ID NO:19.

15. The method of claim 14, wherein the sequence of the oligonucleotide is SEQ ID NO:14.

16. A method of inhibiting poxvirus replication in an individual comprising the steps of administering to the individual an oligonucleotide of between 8 to about 40 nucleotides comprising:
a) an oligoribonucleotide portion which has the sequence of SEQ ID NO: 1; and
b) a flanking region at the 5′ end, the 3′ end or both ends of the oligoribonucleotide portion comprising at least one ribonucleotide, deoxyribonucleotide, modified ribonucleotide or modified deoxyribonucleotide, wherein the oligonucleotide is capable of causing premature termination of transcription of poxvirus genes.

17. The method of claim 16, wherein the oligonucleotide is between 9 and 36 nucleotides long.

18. The method of claim 17, wherein the oligonucleotide is between 9 and 22 nucleotides long.

19. The method of claim 18, wherein the oligonucleotide is between 9 and 13 nucleotides long.

20. The method of claim 16, wherein the sequence of the oligonucleotide is selected from the group consisting of SEQ ID NO:3, SEQ ID NO:5, SEQ ID NO:12, SEQ ID NO:13, SEQ ID NO:14, SEQ ID NO:17, SEQ ID NO:18 and SEQ ID NO:19.

21. The method of claim 20, wherein the sequence of the oligonucleotide is SEQ ID NO: 14.

22. The method of claim 16, wherein the flanking region comprises at least one nucleotide in which the sugar residue is modified at the 2′O position.

23. The method of claim 22, wherein the modification is selected from the group consisting of CH3, CH30, NH2 and CH3CH2NH2.

24. The method of claim 16, wherein the flanking region is selected from the group consisting of phosphorothiols, methylphosphonates, phosphoramidites and morpholinos.

25. The method of claim 16, wherein the oligonucleotide is administered via a route selected from the group consisting of oral, intravenous, intranasal, transdermal, intraperitoneal and rectal.

26. The method of claim 16, wherein the poxvirus is selected from the group consisting of smallpox, Monkeypox, vaccinia virus, cowpox, mouse pox, orf virus and swine pox.

27. The method of claim 16, wherein the individual is a mammal.

28. The method of claim 27, wherein the mammal is selected from the group consisting of a human, a pig and a sheep.

29. The method of claim 28, wherein the mammal is a human.
